(19) United States
(12) Patent Application Publication
(10) Pub. No.: US 2013/0185849 A1
(43) Pub. Date: Jul. 25, 2013

(54) MAGNETIC GOGGLE LENS ATTACHMENT

(71) Applicants: James D. Laughlin, Burlington, VT (US); Jeffrey A. Gilberti, South Burlington, VT (US); Trevor J. Moore, Richmond, VT (US)

(72) Inventors: James D. Laughlin, Burlington, VT (US); Jeffrey A. Gilberti, South Burlington, VT (US); Trevor J. Moore, Richmond, VT (US)

(73) Assignee: The Burton Corporation, Burlington, VT (US)

(21) Appl. No.: 13/357,345

(22) Filed: Jan. 24, 2012

(51) Int. Cl. A61F 9/02

(52) U.S. Cl. 2/431

(57) ABSTRACT

A goggle is disclosed that includes a frame having an opening with a top, a bottom, and opposite sides. A lens may be selectively removable from the opening even when the goggle is worn by an individual. To enable the removal of the lens, the lens may form a magnetic latch with at least one of the top, bottom, or opposite sides of the opening during insertion and removal. The frame may also include an overhang constructed and adapted to promote a pivoting movement of the magnetic latch and positioned over the magnetic latch.

### BACKGROUND

Goggles are used in any number of applications ranging from sports such as skiing and snowboarding to protective eye wear for use in a lab or even a military situation. While each of type of goggle has different design considerations and performance requirements, those requirements may change during use. For example, a snowboarding goggle that may be appropriate for use during a bright day may be too dark when a storm blows in or as evening approaches. Therefore, a single goggle with a single lens may not provide adequate performance for all of the different situations a user may find themselves in.

### SUMMARY

In one embodiment, a goggle may include a frame having an opening and a first lens disposed in the opening that may be selectively removed and replaced with a second lens while the goggle is worn. The selective removal and retention of the lenses may be provided through the use of a magnetic latch including at least one pair of retaining magnets correspondingly located on the goggle frame and lens.

In another embodiment, a goggle may include a frame having an opening with a top, a bottom, and opposite sides. A lens may be selectively removable from the opening. Furthermore, the lens may form a magnetic latch with at least one of the top side, bottom side, left side, or right side of the opening during insertion and removal. The frame may also include an overhang constructed and adapted to promote a pivoting movement of the magnetic latch and positioned over the magnetic latch.

In yet another embodiment, a goggle lens may include a lens and a first magnet coupled to the lens along a first side of the lens. The first magnet may have a first magnetic field direction. The lens may also include a second magnet coupled to the lens along a second side of the lens. The second magnet may have a second field direction that is substantially orthogonal to the first field direction.

In one embodiment, a goggle may include a frame and a first magnet coupled to the frame along a first side of the frame. The first magnet may have a first magnetic field direction. The frame may also include a second magnet coupled to the frame along a second side of the frame. The second magnet may have a second field direction that is substantially orthogonal to the first field direction.

In another embodiment, a goggle may include a frame and a lens. In addition, a first magnet may be coupled to the lens along a first side of the lens with a first field direction. The goggle may also include a second magnet coupled to the lens along a second side of the lens with a second field direction. The second field direction may be substantially orthogonal to the first field direction when the second magnet is coupled to the lens.

In yet another embodiment, a goggle may include a frame having an opening with a first and second side. The first side may include two lips extending along the first side and the second side may include a single lip extending along the second side. The goggle may also include a lens selectively disposed within the opening. Furthermore, the lens may be disposed between the two lips along the first side and it may be flush with the single lip along the second side.

In another embodiment, a method for replacing a goggle lens includes pivoting a lens about a pivot axis defined between a removable lens and a frame. The edge opposite the pivot axis pivots up and out during removal and in and down during insertion relative to a person when in the fitted state.

It should be appreciated that all combinations of the foregoing concepts and additional concepts discussed in greater detail below (provided such concepts are not mutually inconsistent) are contemplated as being part of the inventive subject matter disclosed herein. In addition, all combinations of claimed subject matter are contemplated as being part of the inventive subject matter disclosed herein.

The foregoing and other aspects, embodiments, and features of the present teachings can be more fully understood from the following description in conjunction with the accompanying drawings.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1C present front views of the goggle as the lens is removed from the frame;

FIGS. 2A-2C present isometric views of the goggle as the lens is removed from the frame;

FIGS. 3A-3C present side views of the goggle as the lens is removed from the frame;

FIGS. 4 and 5 present front and rear views of the lens;

FIG. 6 presents a perspective view of the frame detailing the overhanging projection and associated magnet;

FIG. 7 presents a rear perspective view of the lens detailing the magnetic field directions of the attached magnets;

FIGS. 8 and 9 present a front and perspective view of the frame detailing the magnets disposed in the frame;

FIG. 10 presents a perspective view of the frame detailing the lips present along the periphery of the opening in the frame; and

FIG. 11 presents a bottom view of the frame.

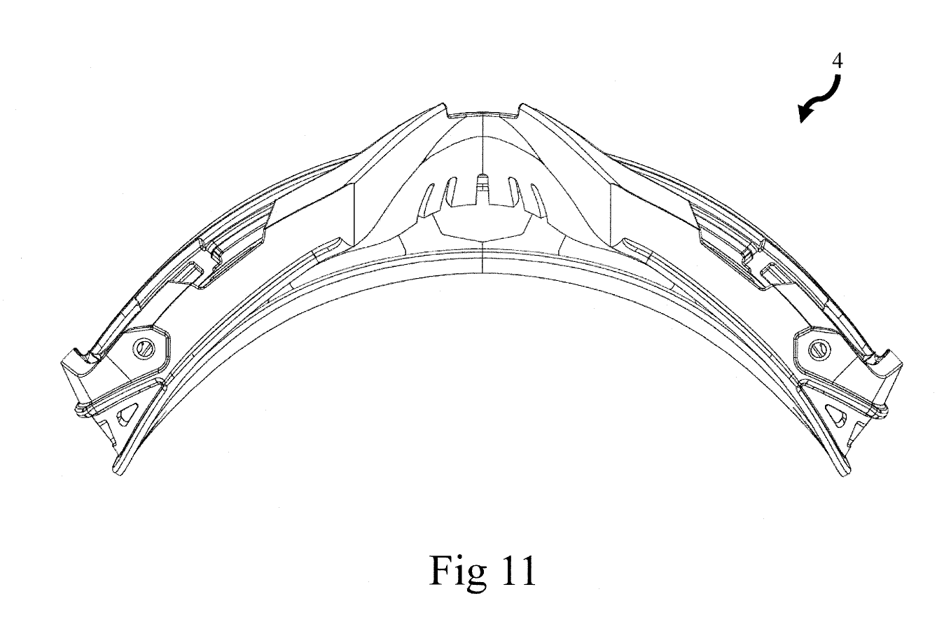

### DETAILED DESCRIPTION

There are any number of instances where a person may want to change the performance of the lens in a goggle. Some reasons to change lenses may include, but are not limited to, changing the tinting for different light conditions, reducing glare, reducing fogging, increasing ballistic/shatter resistance, and/or to use a lens with a different chemical resistance to name a few non-limiting examples. Instead of providing a separate goggle for the different conditions a person may encounter it may be desirable to quickly and simply replace one lens with another with the desired performance characteristics. In view of the above, the inventors have appreciated the need to provide a goggle that may have the lens quickly and easily removed from the frame and replaced with another lens while the goggle is still worn by the person. More specifically, the inventors have recognized the advantages of providing a goggle with a magnetic latch with a corresponding overhang that permits the lens to be pivoted up and outwards to easily and smoothly remove the lens from the frame.

With regards to the disclosure discussed below, it should be understood that there are pairs of mating magnets present on the lens and frame in corresponding locations and orientations. However, for simplicity's sake, magnets located on the frame and lens will be referred to with the same numeral. It should be understood that a magnet present on one component labeled with an identifier will mate with a magnet, or another material attracted to the magnet, present on the other component labeled with the same identifier.

In one embodiment, the goggle 2 includes a frame 4 and lens 8. The frame may include an opening 6 with a top 6a, a bottom 6b, left side 6c, and right side 6d. The lens may be shaped and sized so that it fits within the opening. The lens and frame may include corresponding retaining features such as pairs of magnets located along the periphery of the opening that may attract one another to retain the lens in place during use. These magnet pairs may include corresponding pairs of retaining magnets 12 located along the lower edge of the lens and opening and retaining magnets pairs 14 located on the opposite side along the top edge of the lens and frame opening. Another retaining magnet pair 16 may also be located along a lower edge of the lens and frame opening corresponding to a portion of a nose ridge. In the current embodiment, the various magnets are neodymium magnets. However, it should be understood that the magnets could be any type of magnet including, but not limited to, ceramic, Alnico, other rare earth magnets, and any other applicable magnet suitable for the design requirements of the specific goggle as would be apparent to one of skill in the art.

While the above noted retaining magnets and magnetic latch have been described with respect to mating pairs of magnets the current disclosure is not limited in this fashion. For example, it may be desirable, in some embodiments, to provide a single magnet on the frame or lens with a corresponding material on the other component that is attracted to the magnet. This material may include any magnetically susceptible material, such as iron, nickel, cobalt, magnetic alloys, and other applicable materials as would be apparent to one of skill in the art. For purposes of this patent, magnetic material includes magnets, materials attracted to magnets, and materials with magnetic properties. Therefore, the various magnetic latches, retaining features, and magnetic pairs disclosed herein may include various combinations of magnetic materials. Depending on the application, it may be also be desirable for the materials to be corrosion resistant. Therefore, the material may be a magnetic stainless steel, a ceramic based magnet, or any other corrosion resistant material with magnetic properties.

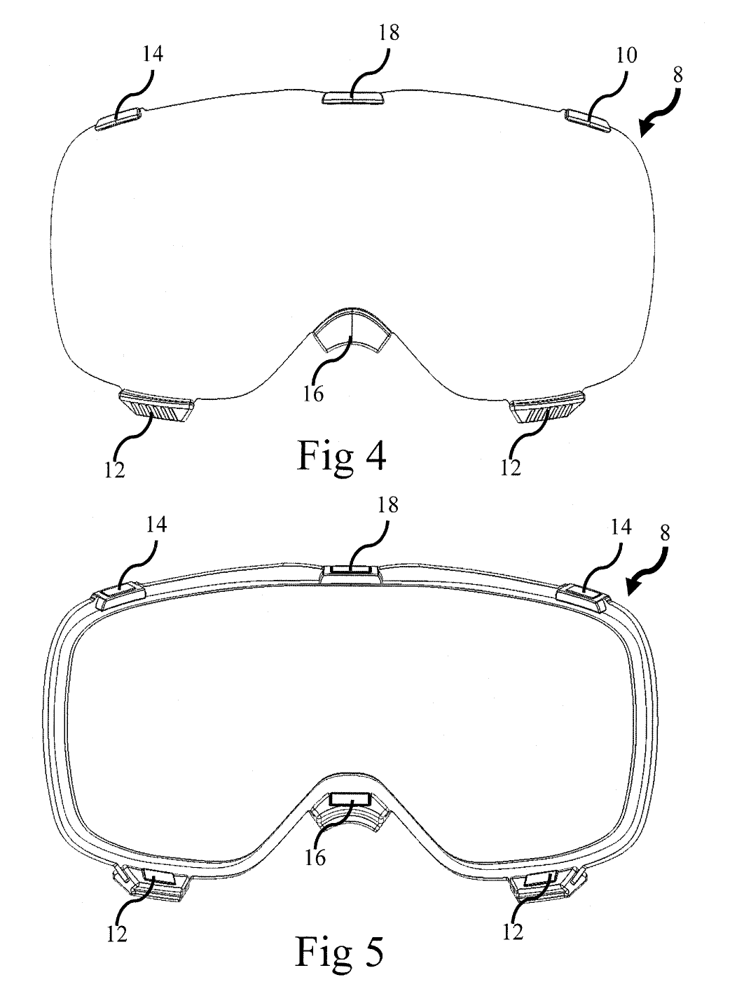

For aesthetic as well as functional purposes, the above noted retaining magnets may be disposed within an outer shell. In some embodiments, the shell may completely encase the magnets. In other embodiments, similar to those depicted in the figures, the shells may expose the mating surfaces of each pair. In addition, the shell may be made from a number of materials including nylon, polyethylene, or other appropriate materials. The shells may also be roughened, coated, knurled, or prepared in any other appropriate manner to facilitate removal of the lens from the frame. In other instances the shells associated with the retaining magnets 12 coupled to the lens 8 may be wedge shaped as seen in FIGS. 4 and 5. It is believed that a wedge shape oriented towards the mating part on the frame may help guide the magnets and lens during insertion. However, other guiding shapes are also envisioned. The shell of the magnets associated with the frame may also include a dovetail, not depicted, that mates with a dovetailed receiving slot on the frame. The magnets may be assembled with the frame by deforming the frame and inserting the dovetail portion of the shell and associated magnet into the slot in the frame. In other embodiments, the coupling may be provided by a mechanical interference fit. An adhesive may also be used in addition to, or in place of, the various mechanical couplings disclosed above.

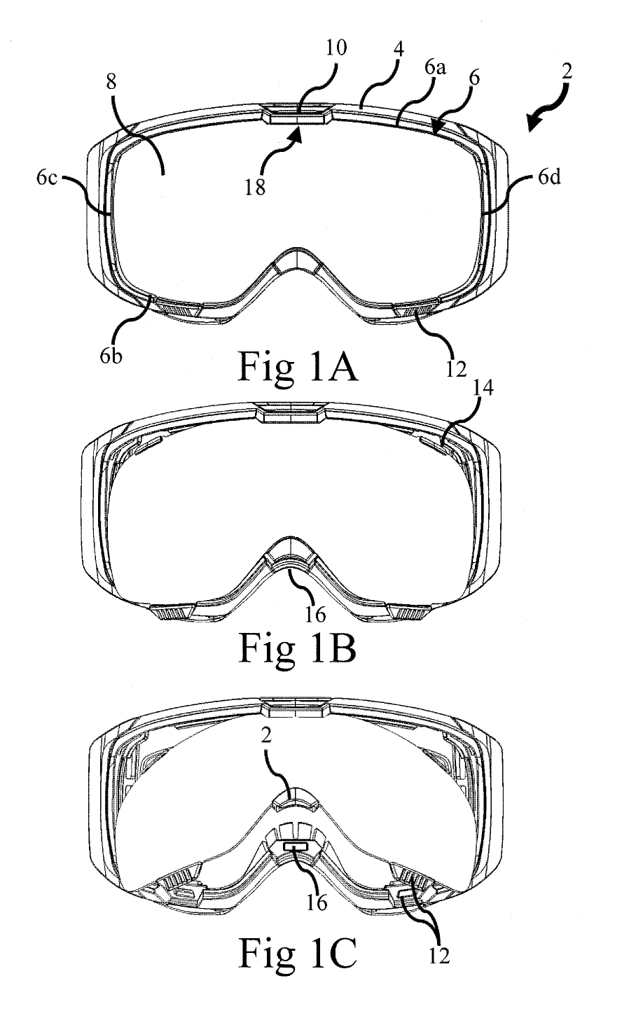

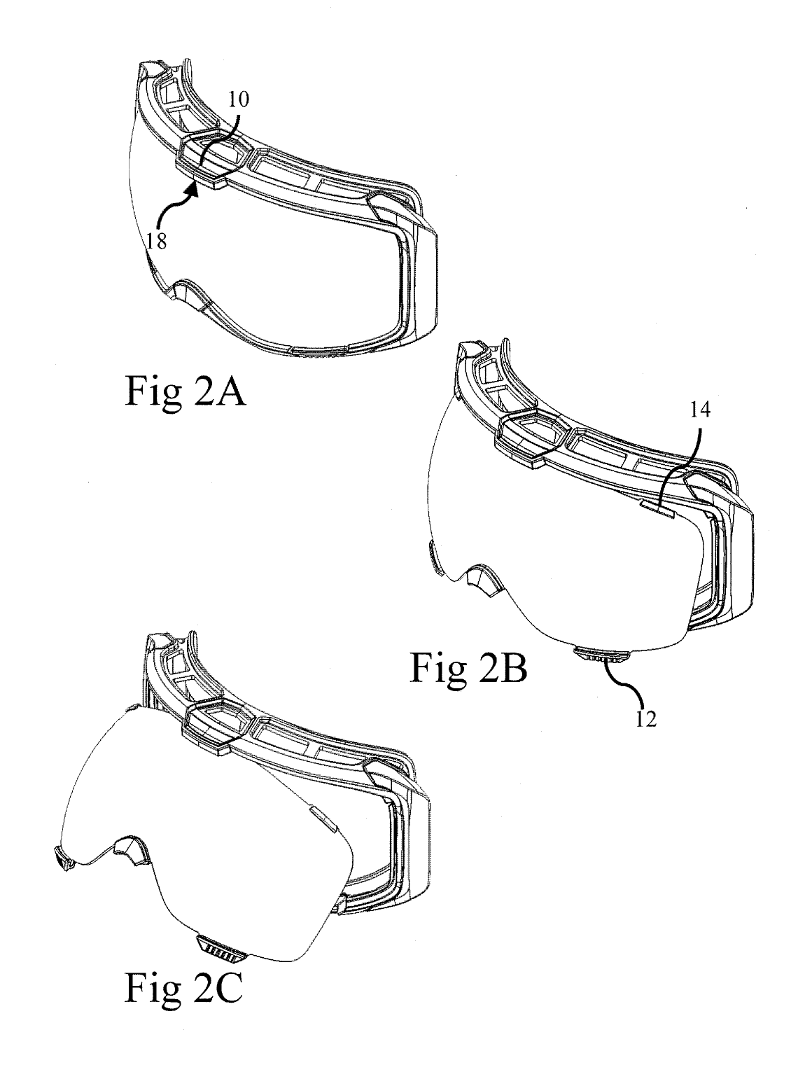

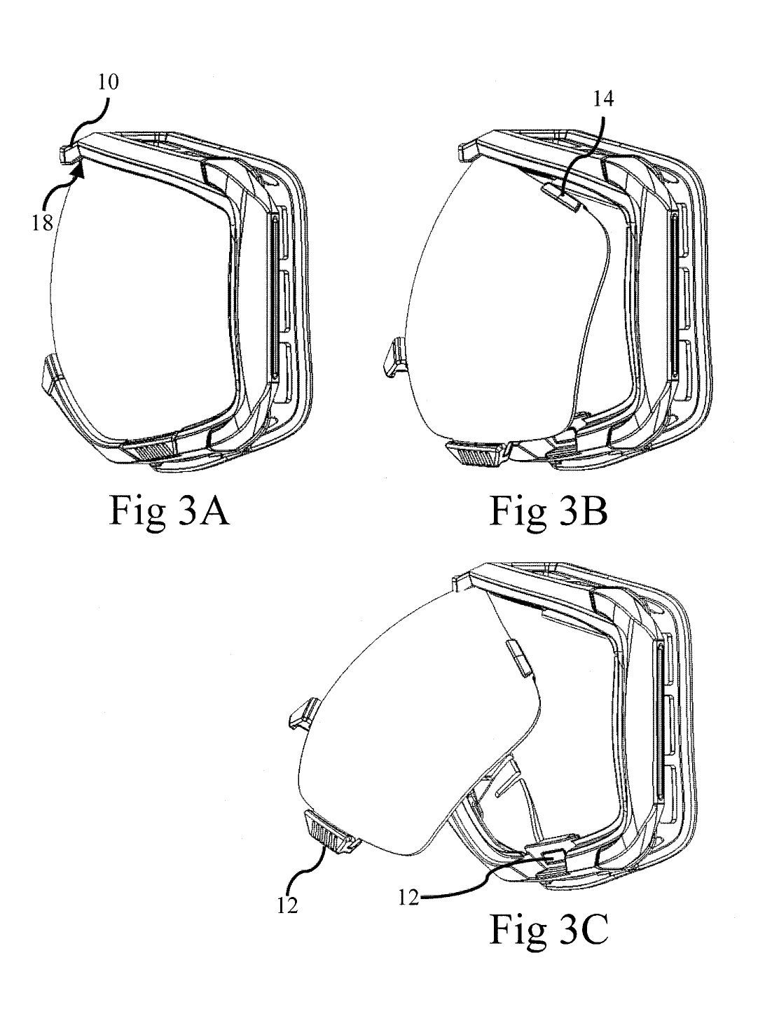

To reduce the removal force of the lens and increase the accuracy of placement, it may be desirable to remove the lens through a pivoting motion about a placement guide. By removing the lens in this manner the retaining force of the magnets may be overcome gradually rather than all at once. For example, if the lens were to pivot upwards the retaining forces on the bottom would be overcome prior to those on the top. Hence, the removal force may be decreased as compared to overcoming the retaining force of every magnet at once. FIGS. 1A-3C generally illustrate the concept of pivoting the lens relative to the goggle frame from several different viewpoints in different states of assembly. As best seen in FIGS. 3A-3C the lens may be pivoted upwards by pushing or prying retaining magnets 12 up and out from the frame. This will disengage the retaining magnets from the bottom edge of the opening and the lens may pivot about the magnetic latch 18.

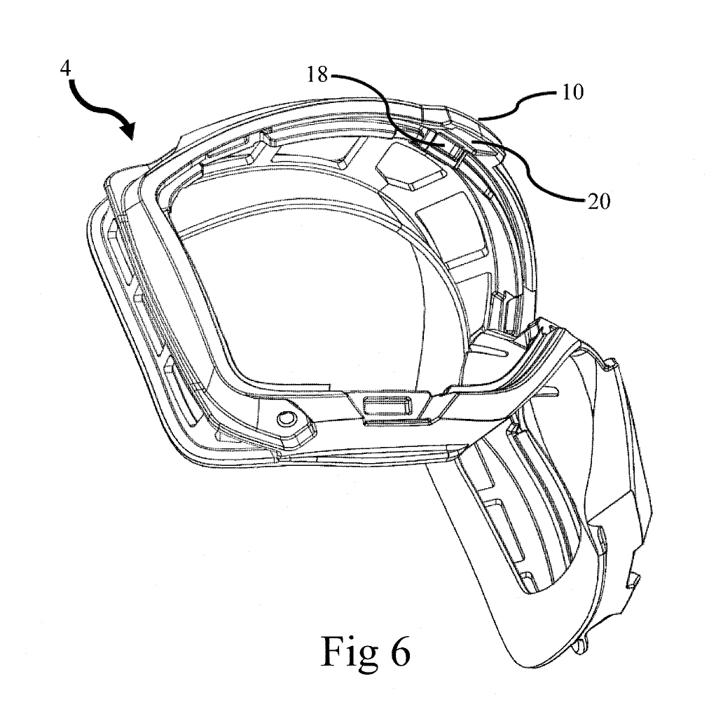

To promote the desired pivoting motion and obtain easy repeatable placement of the lens in the opening in the frame, one or more pairs of magnets may act as a magnetic latch 18 between the frame and lens. To further facilitate the desired pivoting motion of the lens during removal and insertion, the frame may include a projection 10 that overhangs the magnetic latch 18. The portion of the magnetic latch 18 present on the lens may be positioned underneath the overhanging projection during insertion and use. In some embodiments, the overhanging projection may act as a physical guide for placement of the lens and may further promote pivoting of the lens during insertion and removal. As depicted in FIG. 6, in some embodiments the overhanging projection 10 may include an angled surface 20 adjacent to the magnetic latch 18. The angled surface may facilitate the pivoting motion of the lens during insertion and removal by providing a pivot point for the lens to rotate about until the two magnets disengage gradually using the mechanical advantage provided by pivoting the magnet about its edge and onto the angled surface. The angled surface may also act as a ramp intended to guide the portion of the magnetic latch on the lens smoothly towards the corresponding portion of the magnetic latch 18 disposed on the frame during insertion. In a preferred embodiment, the magnets comprising the magnetic latch and the overhanging projection may be located along the top edge of the opening. However, the current disclosure is not limited in this manner and the magnetic latch may be located along any side of the opening.

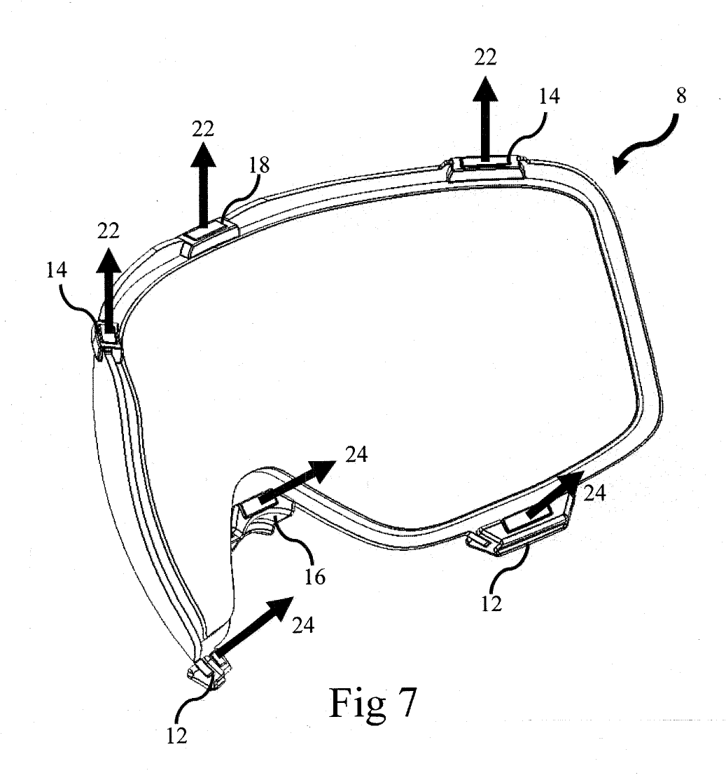

FIG. 7 illustrates the difference in the orientation of the magnetic field direction of the magnets disposed along the periphery of the lens 8. The first magnetic field direction is directed substantially upwards in the figure as depicted by arrows 20 and is associated with the magnetic latch 18 and the retaining magnets 14 disposed along the upper edge of the lens. Without wishing to be bound by theory, while other magnetic field directions may be used, having the magnetic field direction associated with the magnetic latch oriented towards the pivot axis of the lens may reduce the torque applied by magnets 14 and 18 during lens removal. A reduced torque may result in less force being applied to pivot the lens which may help avoid damage to the lens and provide a more controlled feel to removing the lens without the need to pull the magnetic latch apart. Pivoting of the magnetic latch with minimal torque is further facilitated by angled surface 20 discussed above acting as a pivot point to avoid prematurely pulling the magnetic latch apart.

The second magnetic field direction is associated with retaining magnets 12 and 16 disposed along the lower edge of the lens and is depicted by the arrows 24. As shown in FIG. 7, the second magnetic field direction is substantially orthogonal to the first magnetic field direction. In certain embodiments, the first and second magnetic fields may be oriented between approximately 45 to 90 degrees relative to one another. While other orientations are possible, in one embodiment, the second magnetic field direction may also be substantially orthogonal to the pivot axis, e.g. inwards towards a person's face resisting pivoting of the lens about the magnetic latch 18. Without wishing to be bound by theory, orienting the second magnetic field direction orthogonally towards the pivot axis may provide a larger torque to retain the lens within the frame for a given size magnet.

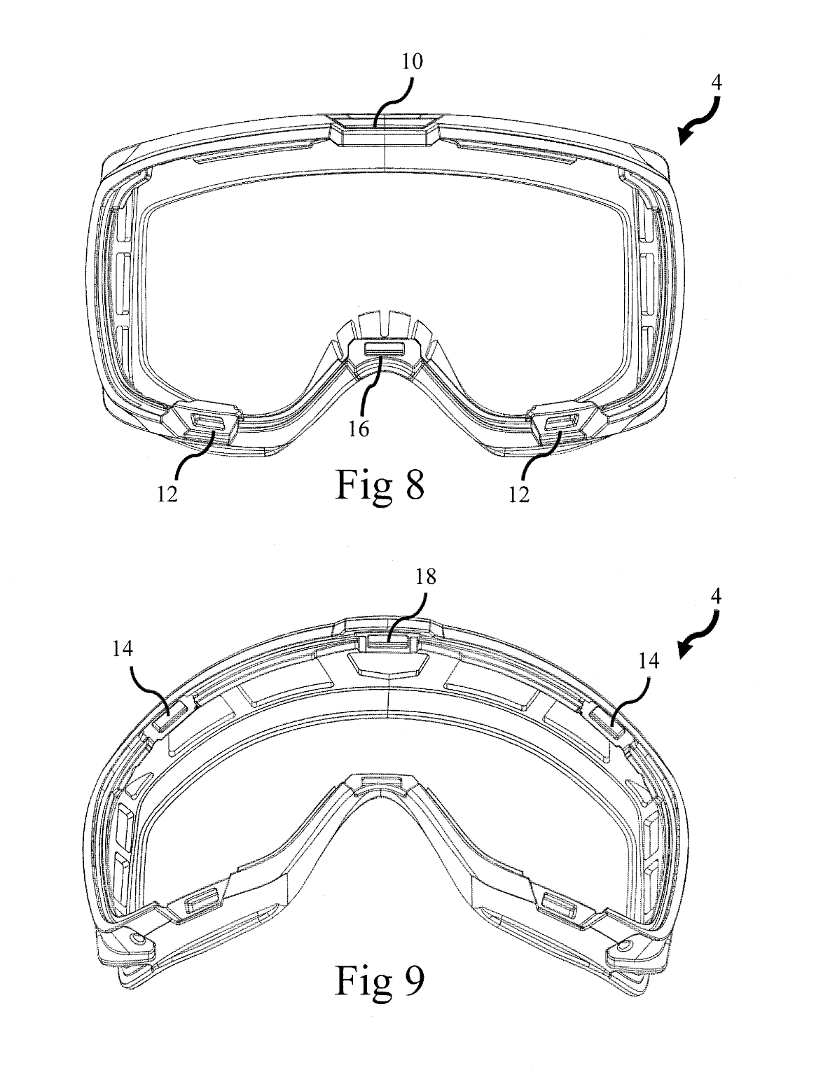

While only the magnetic field directions associated with the magnets on the lens have been shown, the portions of magnet pairs 12, 14, 16, and 18 located on the frame will have corresponding magnetic field directions that are parallel and opposite to the magnetic field directions depicted with respect to the magnets on the lens shown in FIG. 7. Therefore, the magnetic field directions associated with the magnets along the top of the frame opening, i.e. the magnet latch 18 and retaining magnets 14, will be substantially orthogonal to the magnetic field directions associated with retaining magnets 12 and 16 located along the bottom of the frame opening. The locations of the above noted magnets on the frame is shown in FIGS. 8 and 9.

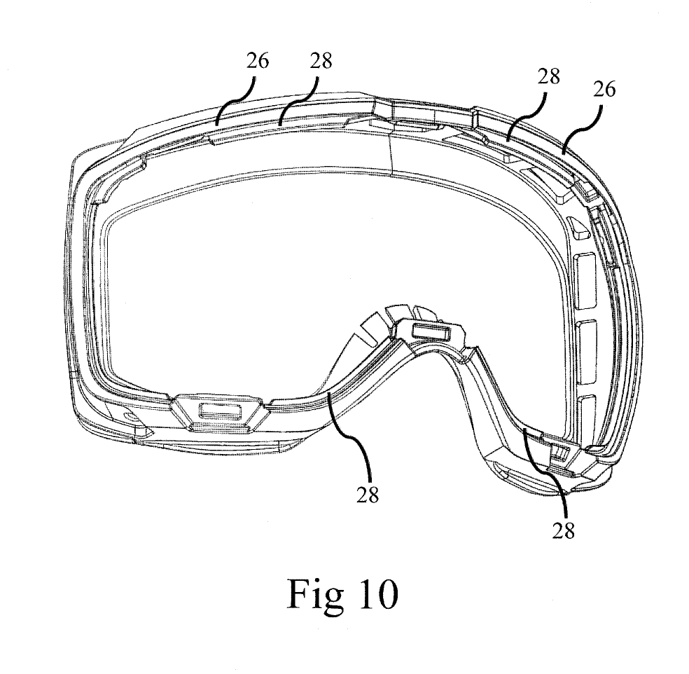

Lenses for a goggle are generally disposed between interior 26 and exterior lips 28 running along the periphery of the opening in the frame to both retain the lens and seal the goggle. In some embodiments, the exterior lip may be flush with an exterior surface of the frame and the interior lip may be recessed from the exterior surface of the goggle. The seal is usually created by pressing the lens between the two lips. However, to provide easy removal of a lens it may be desirable to have at least one side of the opening that does not have an exterior lip. This may permit the lens to easily pivot upwards from at least one edge without being retained by an exterior lip 28. Therefore, in a preferred embodiment, at least one side of the opening may only have an interior lip 26 and it may be located on the side of the frame opposite the location of the magnetic latch. In the embodiment presented in FIG. 10, this corresponds to the bottom side of the frame which only has an interior lip 28. Both top, left, and right edges of the frame include both interior and exterior lips 26 and 28. Since a seal may not be made by pressing the lens between two lips, since only one lip is present along the bottom edge, it may be advantageous to create a seal by holding the lens flush against the interior lip. In one embodiment the retaining magnets 12 and 16 may be mounted to the lens and frame in such a way as to compress a flat surface of the lens against the interior lip to create a seal.

In another embodiment, due to the magnets located along the edge corresponding to the magnetic latch providing an upwards, instead of inwards force, the lens may not be as strongly retained as it is along the opposite edge. Therefore, it may be desirable to provide additional retention force for the lens along the edge corresponding to the location of the magnetic latch by providing both interior and exterior lips along this edge. This is clearly shown in FIG. 10 where the top edge of the opening corresponding to the magnetic latch has both interior and exterior lips 26 and 28. While the interior and exterior lips are shown to include several pieces it is possible that each lip could be a single continuous piece, or there could be a plurality of lips spanning the edge, as the current disclosure is not limited in this fashion.

Although in the embodiments described above the goggle includes a single lens, the goggle may have two or more lenses arranged front to back (as with lenses having an insulating space between lenses to reduce fogging), side to side (as in typical spectacle-type goggles having one lens positioned in front of each eye) and/or other arrangements. Moreover, the frame need not have the particular shape shown in the figures, but instead may have any suitable shape, such as that found in safety glasses, welding goggles, and so on. That is, the frame may accommodate a single unitary lens, or separate lenses, and may have any suitable size, shape or other configuration. The frame also need not include a face gasket or wrap-around shape that closely conforms to the wearer's face, but instead may be formed much like ordinary spectacles. In addition, while the embodiments detailed above describe lenses disposed within an opening of a goggle frame the current disclosure is not limited in this manner. In some embodiments, the lens may be disposed against an exterior edge of the frame such that the lens is placed around the front face of the frame. Alternatively, the lens may simply lie flush against the front face of the frame such that the edges of the frame and lens substantially align with one another. It is also possible an embodiment could contain a combination of the above noted lens and frame arrangements such that different edges of the lens could overlie a frame edge, align with a frame edge, or be disposed within an opening of the frame as the current disclosure is meant to include any of these variations.

The corresponding portions of the retaining magnets and magnetic latch may be mounted to the lens and frame in any suitable way. In this embodiment, the frame and lens may create interference fits with the shells of the retaining magnets and magnetic latch. However, the lens and/or frame may have any suitable features, materials or devices to mount the retaining magnets and magnetic latch. For example, the retaining magnets and magnetic latch may be connected to the frame and/or lens using tabs, holes, pins, locking devices, slots, fasteners, adhesive, friction or snap fit, interference fit, etc.

The goggle may be held in place on a wearer's head in any suitable way. For example, a head support may include an elastic strap. However, the head support may include other features, such as tension adjustment buckles, earstems that extend from the frame to engage the sides and/or ears of a wearer, snaps, clips, hook-and-loop fasteners or other devices to secure the frames to a helmet or other headgear, and so on. In addition to the above, in some embodiments the frame may include adjustable arms located at the sides of the frame that the head support may be attached to. The adjustable arms may allow a user to adjust the fit of the goggle. While several types of head supports have been discussed above, the current disclosure is not limited in this fashion. Instead, the currently disclosed goggle with replaceable lens may be used with any type of head support that allows the goggle to be retained on the head of an individual.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

What is claimed is:

1. A goggle comprising:
a frame having an opening;
a first lens disposed in the opening that may be selectively removed and replaced with a second lens while the goggles are worn; and
a magnetic latch.

2. The goggle of claim 1 wherein the lenses are adapted to pivot about the magnetic latch during removal and insertion into the frame.

3. The goggle of claim 2 wherein an edge opposite a pivot axis between the first lens and frame provided by the magnetic latch pivots up and out during removal relative to a person when in the fitted state.

4. The goggle of claim 1 wherein the magnetic latch comprises a magnetic material.

5. The goggle of claim 1, wherein the lenses are adapted to pivot about an axis substantially orthogonal to a magnetic field direction of the magnetic latch.

6. The goggle of claim 4, further comprising an overhang positioned over the magnetic latch, wherein the lenses are adapted to pivot about a pivot axis defined by the overhang and the magnetic latch.

7. A goggle comprising:
a frame having an opening with a top side, a bottom side, a left side, and a right side; and
a lens that is selectively removable from the opening, wherein the lens forms a magnetic latch with at least one of the top side, bottom side, left side, and right side of the opening during insertion and removal, wherein the frame includes an overhang constructed and adapted to promote a pivoting movement of the magnetic latch and positioned over the magnetic latch.

8. The goggle of claim 7 wherein the magnetic latch comprises a magnetic material.

9. The goggle of claim 7 wherein the overhang includes an angled surface adjacent to the magnetic latch oriented to promote the pivoting movement of the magnetic latch.

10. The goggle of claim 7 wherein the magnetic latch is disposed on the top side of the opening.

11. The goggle of claim 7 wherein the frame further comprises two lips extending along a periphery of the opening on the side corresponding to the magnetic latch and the side opposite the magnetic latch has a single lip extending along the side opposite the magnetic latch.

12. The goggle of claim 7 wherein at least one retaining feature is disposed on at least one of the sides of the opening not corresponding to the magnetic latch.

13. The goggle of claim 12 wherein the retaining feature comprises a magnetic material.

14. The goggle of claim 12 wherein a magnetic field direction of the magnetic latch is substantially orthogonal to a magnetic field direction of the at least one retaining feature.

15. The goggle of claim 14 wherein the angle between the field directions is approximately between 45 to 90 degrees.

16. The goggle of claim 7 wherein a magnetic material is located at or near the magnetic latch.

17. A goggle lens comprising:
a lens;
a first magnet provided along a first side of the lens with a first field direction; and
a second magnet provided along a second side of the lens with a second field direction, wherein the second field direction is substantially orthogonal to the first field direction.

18. The goggle of claim 17 wherein the angle between the first and second field directions is approximately between 45 to 90 degrees.

19. The goggle of claim 17 wherein the first and second magnet are disposed on opposite sides of the lens.

20. A goggle comprising:
a frame;
a lens;
a first magnet provided along a first side of the lens with a first field direction;
a second magnet provided along a second side of the lens with a second field direction, wherein the second field direction is substantially orthogonal to the first field direction;
a first magnetic material coupled to the frame and arranged and oriented to selectively couple with the first magnet coupled to the lens; and
a second magnetic material coupled to the frame and arranged and oriented to selectively couple with the second magnet coupled to the lens.

21. The goggle of claim 20 wherein the angle between the first and second field directions is approximately between 45 to 90 degrees.

22. The goggle of claim 20 wherein the first and second magnet are disposed on opposite sides of the lens.

23. The goggle of claim 20 wherein the first magnet and first magnetic material form a magnetic latch.

24. The goggle of claim 20 wherein the frame includes an overhang constructed and adapted to promote a pivoting movement of the first magnet and first magnetic material and positioned over the first magnetic material.

25. A goggle comprising:
a frame having an opening with a first and second side, wherein the first side includes two lips extending along the first side and the second side includes a single lip extending along the second side; and
a lens selectively disposed within the opening, wherein the lens is disposed between the two lips along the first side and is flush with the single lip along the second side.

26. The goggle of claim 25 wherein the frame and lens further comprise corresponding magnetic materials disposed on the frame and lens that form a magnetic latch.

27. The goggle of claim 26 wherein the first side includes an overhang constructed and adapted to promote a latching movement of the magnetic latch and positioned over the magnetic latch.

28. The goggle of claim 27 wherein the overhang includes an angled surface adjacent to the magnetic latch constructed and arranged to act as a pivot for the magnetic latch.

29. The goggle of claim 25 wherein the first side is a top side of the frame.

30. The goggle of claim 25 wherein two lips extend along left and right sides of the frame.

31. The goggle of claim 25 wherein the first side is a left or right side of the frame.

32. A method for replacing a goggle lens comprising:
pivoting a lens about a pivot axis defined between a removable lens and a frame by a magnetic latch, wherein an edge opposite the pivot axis pivots up and out during removal and in and down during insertion relative to a person when in the fitted state.

33. The method of claim 32, further comprising removing the lens from the frame after the lens abuts an overhanging projection.

34. The method of claim 32, further comprising inserting the lens between an overhanging projection disposed on the frame and the magnetic latch.

35. The method of claim 32 wherein pivoting further comprises pivoting the lens about the pivot axis defined between the removable lens and the frame by the magnetic latch comprising a magnetic material.
